(19) United States
(12) Patent Application Publication
(10) Pub. No.: US 2026/0203894 A1
(43) Pub. Date: Jul. 16, 2026

(54) Mammography Device Outputs for Broad System Compatibility

(71) Applicant: Google LLC, Mountain View, CA (US)

(72) Inventors: Amirhossein Kiani, San Francisco, CA (US); Rory Sayres, Redwood City, CA (US); Marc Peter Tarca Wilson, London (GB); Rubin Chen Zhang, San Francisco, CA (US); Atilla Peter Kiraly, San Jose, CA (US); Ivan Protsyuk, London (GB); Megumi Morigami, St Albans (GB); Thidanun Saensuksopa, San Francisco, CA (US); Tiya Ann Tiyasirichokchai, New York, NY (US)

(21) Appl. No.: 19/135,217

(22) PCT Filed: Dec. 6, 2022
(86) PCT No.: PCT/US2022/051969

§ 371 Date: Jun. 3, 2025

(51) Int. Cl. G06T 7/00; A61B 6/46; A61B 6/50; G06V 10/25; G06V 10/26; G06V 10/764

(52) U.S. Cl. CPC G06T 7/0012; A61B 6/463; A61B 6/502; G06V 10/25; G06V 10/26; G06V 10/764; G06T 2200/24; G06T 2207/10116; G06T 2207/20081; G06T 2207/30068; G06T 2207/30096; G06V 2201/03

(57) ABSTRACT

Systems and methods for providing a visual representation output descriptive of a preliminary diagnosis can include obtaining radiograph data, processing the radiograph data with a machine-learned model to generate one or more classification outputs, and generating the visual representation output based on the one or more classification outputs. The visual representation output can be generated such that the visual representation output can be provided for display on a plurality of different display types with a plurality of different technical capabilities.

### FIELD

The present disclosure relates generally to providing a mammography device output. More particularly, the present disclosure relates to processing mammography image data with a machine-learned model to generate localization and classification outputs, which can then be utilized to generate a visual representation output to be provided for display.

### BACKGROUND

Medical imaging viewing and processing can rely on expensive specialized hardware that may have limited utility outside of that specific type of medical image viewing. Additionally and/or alternatively, the transmission of this data across a network for viewing on other displays can be limited based on the specialized hardware required. Such specialization can lead to large costs and/or limited spaces for viewing and processing.

The computational power required for artificial intelligence processing can be relatively large compared to the normal functions of a computing device. Therefore, in order to consistently perform such processing tasks, computing devices with high technical capabilities may be relied upon when performing such actions. The cost of having high capability computing devices can be expensive, and providing a dedicated high capability computing device with each imaging device (e.g., an on-premises device for each imaging device) can cause exponential growth in that cost. Additionally, for the time in which the imaging device is not being used, the utility of the processing power of the high capability devices may be left unutilized.

### SUMMARY

Aspects and advantages of embodiments of the present disclosure will be set forth in part in the following description, or can be learned from the description, or can be learned through practice of the embodiments.

One example aspect of the present disclosure is directed to a computing system for providing a mammography device output. The system can include one or more processors and one or more non-transitory computer-readable media that collectively store instructions that, when executed by the one or more processors, cause the computing system to perform operations. The operations can include obtaining mammography image data. The mammography image data can include radiograph data associated with an individual. In some implementations, the mammography image data can be descriptive of one or more radiograph images. The operations can include processing the mammography image data with a machine-learned model to generate one or more classification outputs. The machine-learned model may have been trained to detect and classify an abnormality in the radiograph data. The abnormality can be associated with a predicted cancer classification. In some implementations, the one or more classification outputs can be descriptive of one or more detected abnormalities. The operations can include generating a visual representation output based on the mammography image data and the one or more classification outputs. The visual representation output can include one or more annotated radiograph images. In some implementations, the one or more annotated radiograph images can be descriptive of the one or more radiograph images with one or more annotations associated with the one or more detected abnormalities. The visual representation output can include text data associated with the one or more classification outputs. The operations can include storing the visual representation output in a medical image output file.

In some implementations, the one or more annotations can indicate one or more portions of the one or more radiograph images determined to include a region of interest. The text data can include text information descriptive of an abnormality classification. The abnormality classification can be descriptive of the one or more radiograph images including pixel data associated with a tissue abnormality. In some implementations, the text data can include text information descriptive of a number of detected abnormalities. The one or more radiograph images can include a first bit depth. The one or more annotated radiograph images can include a second bit depth. The second bit depth can be smaller than the first bit depth.

In some implementations, processing the mammography image data with the machine-learned model to generate the one or more classification outputs can include cloud computing. The operations can include transmitting the medical image output file to a picture archiving and communication system and providing the visual representation output for display via a display computing device. The medical image output file can be configured to instruct the display computing device to display the visual representation output within a set grayscale range.

In some implementations, the one or more annotations can include one or more boxes. The one or more boxes can indicate one or more regions of interests associated with one or more detected abnormalities. A size, a width, and a length of the one or more annotations can be determined based on determined dimensions of the one or more detected abnormalities. In some implementations, the mammography image data can be descriptive of a left mediolateral oblique view, a right mediolateral oblique view, a left craniocaudal view, and a right craniocaudal view.

Another example aspect of the present disclosure is directed to one or more non-transitory computer-readable media that collectively store instructions that, when executed by one or more computing devices, cause the one or more computing devices to perform operations. The operations can include obtaining mammography image data. The mammography image data can include radiograph data associated with an individual. In some implementations, the mammography image data can be descriptive of one or more radiograph images. The operations can include processing the mammography image data with a machine-learned model to generate one or more classification outputs. The machine-learned model may have been trained to detect and classify an abnormality in the radiograph data. In some implementations, the abnormality can be associated with a predicted cancer classification. The one or more classification outputs can be descriptive of one or more detected abnormalities. The operations can include processing the one or more radiograph images to generate one or more presentation images. The one or more presentation images can be descriptive of the one or more radiograph images augmented for presentation on a visual display. The operations can include generating a visual representation output based on the one or more presentation images and the one or more classification outputs. In some implementations, the visual representation output can include one or more annotated radiograph images. The one or more annotated radiograph images can be descriptive of the one or more presentation images with one or more annotations associated with the one or more detected abnormalities. The operations can include providing the visual representation output to a display computing system for display.

In some implementations, the machine-learned model can include a feature detection model trained to identify regions of interest associated with abnormal features in a radiograph. The feature detection model can generate one or more bounding boxes. The machine-learned model can include a segmentation model trained to process a radiograph and segment a region of interest from the radiograph. In some implementations, the machine-learned model can include a classification model trained to process a region of interest to generate an abnormality classification. The operations can include storing the mammography image data and the visual representation output in association with one or more individual identifiers that identify the individual. The mammography image data and the visual representation output can be stored in a medical image output file. The medical image output file can be formatted to provide the one or more radiograph images and the visual representation output in a displayable format for a plurality of display types. In some implementations, the operations can include presenting a title slide. The title slide can include one or more instructions. The operations can include presenting the one or more radiograph images via the user interface, in response to obtaining the mammography image data. The visual representation output can be provided for display via the user interface.

Another example aspect of the present disclosure is directed to a computer-implemented method for providing a mammography device output. The method can include obtaining, by a cloud computing system including one or more processors, mammography image data. The mammography image data can include x-ray data associated with breast tissue of an individual. In some implementations, the mammography image data can be descriptive of one or more radiograph images. The method can include processing, by the cloud computing system, the mammography image data with a machine-learned model to generate one or more classification outputs. The machine-learned model may have been trained to detect and classify an abnormality in the x-ray data. In some implementations, the abnormality can be associated with a predicted cancer classification. The one or more classification outputs can be descriptive of one or more detected abnormalities. The method can include generating, by the cloud computing system, a visual representation output based on the mammography image data and the one or more classification outputs. The visual representation output can include one or more annotated radiograph images. The one or more annotated radiograph images can be descriptive of the one or more radiograph images with one or more annotations associated with the one or more detected abnormalities. The method can include providing, by the cloud computing system, the visual representation output to a picture archiving and communications system.

In some implementations, the cloud computing system can include one or more server computing systems. The one or more server computing systems can include the one or more processors. The operations can include transmitting, from the picture archiving and communications system to a display device, the visual representation output. In some implementations, the one or more radiograph images can include a plurality of pixels in a first range of at least one of 1024 shades of gray or 4096 shades of gray. The one or more annotated radiograph images can include pixel data in a second range. The second range can include less shades than the first range.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1A depicts a block diagram of an example computing system that performs visual representation output generation according to example embodiments of the present disclosure.

FIG. 1B depicts a block diagram of an example computing device that performs visual representation output generation according to example embodiments of the present disclosure.

FIG. 1C depicts a block diagram of an example computing device that performs visual representation output generation according to example embodiments of the present disclosure.

FIGS. 2A-2C depict illustrations of example user interface outputs and original data according to example embodiments of the present disclosure.

FIG. 3 depicts a block diagram of an example visual representation generation model according to example embodiments of the present disclosure.

FIG. 4 depicts a block diagram of an example machine-learned model training according to example embodiments of the present disclosure.

FIG. 5 depicts a block diagram of an example output generation and display system according to example embodiments of the present disclosure.

FIG. 6 depicts a flow chart diagram of an example method to perform medical image output file generation and storage according to example embodiments of the present disclosure.

FIG. 7 depicts a flow chart diagram of an example method to perform visual representation output generation according to example embodiments of the present disclosure.

FIG. 8 depicts a flow chart diagram of an example method to perform visual representation output generation according to example embodiments of the present disclosure.

Reference numerals that are repeated across plural figures are intended to identify the same features in various implementations.

### DETAILED DESCRIPTION

### Overview

Generally, the present disclosure is directed to systems and methods for providing a mammography device output. In particular, the systems and methods disclosed herein can leverage one or more machine-learned models to identify and classify abnormal features of an input (e.g., one or more Digital Imaging and Communications in Medicine (DICOM) images), which can be utilized to generate a visual representation output for display. The systems and methods disclosed herein can provide medical professionals and/or patients with a descriptive representation of a preliminary identification of potentially cancerous abnormalities in a format that is universally displayable across picture archiving and communication systems (PACS) and easily understandable. For example, the systems and methods can include obtaining mammography image data. The mammography image data can include radiograph data associated with an individual. In some implementations, the mammography image data can be descriptive of one or more radiograph images. The systems and methods can include processing the mammography image data with a machine-learned model to generate one or more classification outputs. The machine-learned model may have been trained to detect and classify an abnormality in the radiograph data. In some implementations, the abnormality can be associated with a predicted cancer classification. The one or more classification outputs can be descriptive of one or more detected abnormalities. The systems and methods can include generating a visual representation output based on the mammography image data and the one or more classification outputs. The visual representation output can include one or more annotated radiograph images in a DICOM format. The one or more annotated radiograph images can be descriptive of the one or more radiograph images with one or more annotations associated with the one or more detected abnormalities. In some implementations, the visual representation output can include text data associated with the one or more classification outputs. The systems and methods can include providing the visual representation output to a display computing system for display.

When utilizing machine-learned models for medical imaging, communicating the output in a meaningful and efficient format can be important for the interpretation process. For example, communicating results in a streamlined manner that does not distract the user and reduces the chance of misunderstanding can provide a more intuitive process. Additionally and/or alternatively, when utilizing the systems and methods disclosed herein, the system upon which visual representations surface may provide a more convenient system for users to access data (e.g., to access machine-learned model outputs). Early versions of Computer Aided Detection (CADe) for mammography had multiple false positive findings and required a special device to access resulting in multiple pain points for users. The systems and methods disclosed herein can present a solution to minimize usage difficulties with minimal interference with existing workflows and systems.

The systems and methods can obtain mammography image data. The mammography image data can include radiograph data associated with an individual. In some implementations, the mammography image data can be descriptive of one or more radiograph images. The mammography image data can be descriptive of a left mediolateral oblique view, a right mediolateral oblique view, a left craniocaudal view, and a right craniocaudal view. Additionally and/or alternatively, one or more other views may be included in the mammography image data.

The mammography image data can be processed with a machine-learned model to generate one or more classification outputs. The machine-learned model may have been trained to detect and classify an abnormality in the radiograph data. The abnormality can be associated with a predicted cancer classification. In some implementations, the one or more classification outputs can be descriptive of one or more detected abnormalities. Additionally and/or alternatively, processing the mammography image data with the machine-learned model to generate the one or more classification outputs can include cloud computing. In some implementations, the machine-learned model can process the mammography image data to identify regions of interest, segment the regions of interest, and classify the features in the regions of interest. The regions of interest may be identified by a detection model trained to determine one or more features for classification. The region encompassing the feature can be segmented then classified. The machine-learned model can include one or more detection models, one or more segmentation models, one or more classification models, and/or one or more semantic understanding models. The machine-learned model may be trained on a training dataset comprising a plurality of training radiograph images associated with breast tissue of an individual. The plurality of training radiograph images may include ground truth healthy images and/or ground truth cancer images (e.g., images with one or more abnormal features associated with cancer cells). The plurality of datasets may be augmented through various random image manipulations (skewing, stretching, etc.) such that a single image is run multiple times with each distortion for either inference or training. The training dataset may include a plurality of ground truth labels and/or a plurality of example segmentation masks.

A visual representation output can then be generated based on the mammography image data and the one or more classification outputs. The visual representation output can include one or more annotated radiograph images. The one or more annotated radiograph images can be descriptive of the one or more radiograph images with one or more annotations associated with the one or more detected abnormalities. Additionally and/or alternatively, the visual representation output can include text data associated with the one or more classification outputs. The one or more annotations can be specifically generated for each respective detected abnormality. For example the size and/or shape of each annotation may be specific to each respective detected abnormality. In some implementations, the size and/or shape may be proportional to the size and/or shape of the detected abnormal lesion. The one or more annotations can include two superimposed rectangles of contrasting intensities (light and dark) with the second having thicker borders. The light intensity can be selected to be brighter than the brightest component in any grayscale image and similarly the darkest intensity can be selected to be darker than the darkest intensity in the grayscale image. These contrasting intensities can allow for the region to be clearly marked regardless of the underlying grayscale image and can remain visible as users adjust the display window/level/contrast. In some implementations, the visual representation output can include one or more grayscale images.

In some implementations, the one or more annotations can indicate one or more portions of the one or more radiograph images determined to include a region of interest. The text data can include text information descriptive of an abnormality classification. The abnormality classification can be descriptive of the one or more radiograph images including pixel data associated with a tissue abnormality. Additionally and/or alternatively, the text data can include text information descriptive of a number of detected abnormalities. The text data can include a text label indicating the breast where the image came from (left or right) and/or the artificial intelligence assessment result for that breast (e.g., a binary score computed from this image and/or another image from the same breast: normal or abnormal). A breast may be considered abnormal if the machine-learned model reports at least one region of interest, with the risk score above the threshold on any image of that breast. Otherwise, the breast may receive a normal score with no regions highlighted. The text data may include a text warning indicating that the visual representation output is not an original image and should not be used alone for making clinical decisions. Additionally and/or alternatively, the text data can include a text label indicating the number of regions of interest on a given image and/or other images of the same breast. In some implementations, the one or more annotations can include one or more boxes rendered over an individual's radiograph image. The one or more boxes can indicate one or more regions of interest associated with one or more detected abnormalities. In some implementations, a size, a width, and a length of the one or more annotations may be determined based on determined dimensions of the one or more detected abnormalities. In some implementations, the shape of the annotations may be ovals instead of boxes.

The visual representation output can then be provided to a display computing system for display. The display computing system may include a general purpose display (e.g., a standard light-emitting diode display, an organic light-emitting diode display, a touch screen monitor, a plasma screen monitor, and/or another monitor type). Alternatively and/or additionally, the display computing system may include a specialist mammography display. The visual representation output may be of a different color range than the one or more radiograph images.

In some implementations, the one or more radiograph images can include a first grayscale color palette (e.g., 4096 possible shades of gray (12-bit) or 1024 possible shades of gray). The one or more annotated radiograph images can include a second grayscale palette (e.g., 256 possible shades of gray). Additionally and/or alternatively, the second possible shades of gray for the second grayscale palette can be less than the first possible shades of gray for the first grayscale palette. The second number may be optimized for universal display on a plurality of different display types. For example, the raw radiograph images and the annotated images of the visual representation output may have different grayscale color palettes such that the output can be provided for display on displays that may not be optimized for raw medical images. For example, the raw radiographic images and the annotated images would have different bit-depths (e.g., 12-bit vs. 8-bit).

In some implementations, the systems and methods can transmit the visual representation output to a picture archiving and communication system for storage. The picture archiving and communication system can archive the mammography image data and/or the visual representation output. The archived data can then be obtained and displayed in response to one or more requests. The archived data may be accessed via a plurality of computing devices across a network.

The systems and methods may include universal display conversion of the output to enable the display of the output on displays that may not be optimized for mammography radiograph images. In some implementations, the systems and methods can utilize hanging protocols to adjust one or more of the images to be displayed in a particular order. The hanging protocols can be utilized to generate a user experience that can provide instructions for display, then the unannotated radiograph images, and then the annotated images. For example, the systems and methods can include obtaining mammography image data. The mammography image data can include radiograph data associated with an individual. In some implementations, the mammography image data can be descriptive of one or more radiograph images. The systems and methods can include processing the mammography image data with a machine-learned model to generate one or more classification outputs. The machine-learned model may have been trained to detect and classify an abnormality in the radiograph data. The abnormality can be associated with a predicted cancer classification. In some implementations, the one or more classification outputs can be descriptive of one or more detected abnormalities. The systems and methods can include processing the one or more radiograph images to generate one or more presentation images. The one or more presentation images can be descriptive of the one or more radiograph images augmented for presentation on a visual display. The systems and methods can include generating a visual representation output based on the one or more presentation images and the one or more classification outputs. The visual representation output can include one or more annotated radiograph images. In some implementations, the one or more annotated radiograph images can be descriptive of the one or more presentation images with one or more annotations associated with the one or more detected abnormalities. Additionally and/or alternatively, the systems and methods can include providing the visual representation output to a display computing system for display.

The systems and methods can obtain mammography image data. The mammography image data can include radiograph data associated with an individual. In some implementations, the mammography image data can be descriptive of one or more radiograph images. The one or more radiograph images can be descriptive of an x-ray of one or more angles of an individual's breast. The mammography image data may include metadata associated with the user, which may include an individual's age, weight, and/or other biological information.

The mammography image data can be processed with a machine-learned model to generate one or more classification outputs. The machine-learned model may have been trained to detect and classify an abnormality in the radiograph data. The abnormality can be associated with a predicted cancer classification. The one or more classification outputs can be descriptive of one or more detected abnormalities. The one or more classification outputs can be associated with a preliminary diagnosis associated with processed radiograph data (e.g., a preliminary cancer diagnosis). In some implementations, the machine-learned model can be trained to process and provide a preliminary diagnosis associated with one or more other body parts and/or one or more other diagnostic options (e.g., a preliminary benign cyst diagnosis, a preliminary anterior cruciate ligament (ACL) tear diagnosis for a knee, and/or a stroke by processing brain imagery). The systems and methods may generate an optimized output based on the machine-learned model output that can effectively communicate a preliminary diagnosis to a radiologist, another medical professional, an insurance provider, and/or a patient.

In some implementations, the machine-learned model can include a feature detection model trained to identify regions of interest associated with abnormal features in a radiograph. The feature detection model can generate one or more bounding boxes. In some implementations, the machine-learned model can include a segmentation model trained to process a radiograph and segment a region of interest from the radiograph. Additionally and/or alternatively, the machine-learned model can include a classification model trained to process a region of interest to generate an abnormality classification.

The one or more radiograph images can be processed to generate one or more presentation images. The one or more presentation images can be descriptive of the one or more radiograph images augmented for presentation on a visual display. In some implementations, pixels of the bounding boxes detected by the feature detection model may be encoded using a lightest pixel value of a grayscale range, e.g. a maximum pixel value. Using the lightest pixel value to encode the pixels of the bounding box can ensure that the bounding box remains clearly visible whatever the color/grayscale resolution of the display device. The maximum pixel value may be the maximum pixel value of a specialist mammography display (e.g. a display with a 1024 level grayscale range). This can ensure that bounding boxes in the one or more presentation images are viewable on both specialist mammography displays and generic computer displays.

A visual representation output can then be generated based on the one or more presentation images and the one or more classification outputs. The visual representation output can include one or more annotated radiograph images. In some implementations, the one or more annotated radiograph images can be descriptive of the one or more presentation images with one or more annotations associated with the one or more detected abnormalities.

The visual representation output can then be provided to a display computing system for display. The display computing system may be configured to provide 256-levels of grayscale. The display computing system can include a display device that may not be specialized for radiograph data and may be utilized for one or more other display tasks. The visual representation output can include a plurality of slides that may be navigated through automatically at a given time interval and/or via one or more user inputs.

In some implementations, the systems and methods can store the mammography image data and the visual representation output in association with one or more individual identifiers that identify the individual.

Additionally and/or alternatively, the system and methods can present a set of instructions and/or a set of warnings (e.g., a title slide) via an image and/or structured text. The title slide can include one or more instructions. In response to obtaining the mammography image data, the systems and methods may present the one or more radiograph images via the user interface. In some implementations, the visual representation output can be provided for display via the user interface.

Additionally and/or alternatively, the systems and methods can utilize a cloud computing system to utilize the processing power of one or more server computing systems. The cloud computing system can be utilized in order to complement and/or replace a dedicated workstation for mammography processing. For example, the systems and methods can include obtaining, by a cloud computing system, mammography image data. The mammography image data can include x-ray data associated with breast tissue of an individual. In some implementations, the mammography image data can be descriptive of one or more radiograph images. The systems and methods can include processing, by the cloud computing system, the mammography image data with a machine-learned model to generate one or more classification outputs. The machine-learned model may have been trained to detect and classify an abnormality in the radiograph data, wherein the abnormality is associated with a predicted cancer classification. The one or more classification outputs can be descriptive of one or more detected abnormalities. The systems and methods can include generating, by the cloud computing system, a visual representation output based on the mammography image data and the one or more classification outputs. The visual representation output can include one or more annotated radiograph images. In some implementations, the one or more annotated radiograph images can be descriptive of the one or more radiograph images with one or more annotations associated with the one or more detected abnormalities. The systems and methods can include providing, by the cloud computing system, the visual representation output to a picture archiving and communications system.

A cloud computing system can obtain mammography image data. The mammography image data can include x-ray data associated with breast tissue of an individual. In some implementations, the mammography image data can be descriptive of one or more radiograph images. The cloud computing system can include one or more server computing systems. The one or more server computing systems can include the one or more processors. The one or more radiograph images can be digital imaging and communications in medicine standard (DICOM) images.

The cloud computing system can process the mammography image data with a machine-learned model to generate one or more classification outputs. The machine-learned model may be trained to detect and classify an abnormality in the x-ray data. The abnormality can be associated with a predicted cancer classification. In some implementations, the one or more classification outputs can be descriptive of one or more detected abnormalities. The mammography image data may be processed in black-and-white (e.g., in grayscale). The mammography image data can include one or more high fidelity images that may be obtained and/or stored in a central computing system.

The cloud computing system can generate a visual representation output based on the mammography image data and the one or more classification outputs. The visual representation output can include one or more annotated radiograph images. The one or more annotated radiograph images can be descriptive of the one or more radiograph images with one or more annotations associated with the one or more detected abnormalities. The visual representation may include only positive abnormality classification and may remove and/or determine not to include any annotations associated with negative classifications for one or more determined regions of interest. In some implementations, the visual representation output can include text descriptive of a number of detected abnormalities and/or one or more region of interest classifications and/or one or more image classifications. The systems and methods may generate a visual representation output for each obtained radiograph image of a plurality of radiograph images. In some implementations, the annotations may be overlapping, which may indicate overlapping regions of interest. Alternatively and/or additionally, the annotation associated with a higher confidence score of abnormality may be displayed with the other annotation removed. Alternatively and/or additionally, the larger annotation associated with a larger bounding box may be kept. The visual representation output may be generated based on a particular resolution, bit depth, and/or color range associated with one or more hardware devices.

The cloud computing system can then provide the visual representation output to a picture archiving and communications system. The visual representation output can be optimized for the picture archiving and communications system. The visual representation output can be generated to be universally compatible for a plurality of different types of display. The systems and methods may be utilized to provide a preliminary diagnosis without the cost of a dedicated high processing power workstation for each individual testing unit. The systems and methods may utilize cloud computing to allow the use of the processing power of one or more server computing systems, which may lead to less costs and less dedicated processing components and/or devices. The metadata for the mammography image data may be utilized for uniform posing of the outputs (e.g., corrected orientation for uniformity based on hanging protocols). The picture archiving and communications system can include a plurality of computing devices with different displays, different interfaces, and/or technical capabilities.

In some implementations, hanging protocols in picture archiving and communications systems can allow for the images in a dataset (e.g., a study and/or a group of images for a particular individual) to be grouped and/or oriented in a particular fashion. The hanging protocol may cause the system to turn an image to keep a consistent view for radiologists. The protocols can allow one to step through different viewing configurations and also define their own based on personal preferences.

In some implementations, the cloud computing system can transmit, from the picture archiving and communications system to a display device, the visual representation output.

Additionally and/or alternatively, the one or more radiograph images can be stored in 1024 shades of gray (e.g., the one or more radiograph images can include a plurality of pixels in a first range of 1024 shades of gray or 10-bit depth), and the one or more annotated radiograph images can use another bit depth (e.g., the one or more annotated radiograph images can include pixel data in an 8-bit depth). The second grayscale palette and/or second bit depth can include less shades of gray than the first grayscale palette and/or first bit depth.

In some implementations, the visual representation output can be stored in a medical image output file (e.g., a mammography device output file). The medical image output file can be configured to instruct a display computing device to display the visual representation output within a set grayscale range. Additionally and/or alternatively, the mammography image data and the visual representation output can be stored in a medical image output file. The medical image output file can be formatted to provide the one or more radiograph images and the visual representation output in a displayable format for a plurality of display types. In some implementations, the medical image output file can be formatted to provide a set of slides for display. For example, a title slide (e.g., a slide with instructions for interpreting and/or understanding the slides), a clean medical image slide (e.g., one or more medical images without annotations), and an annotated medical image slide (e.g., the visual representation output) may be stored for display upon request. Alternatively and/or additionally, the medical image output file can be formatted as a plurality of images. In some implementations, the medical image output file can be formatted as a plurality of compressed medical images that may be displayable in a broad assortment of applications and/or a broad variety of display types.

The systems and methods of the present disclosure provide a number of technical effects and benefits. As one example, the system and methods can provide a preliminary diagnosis to a medical professional and/or a patient. More specifically, the systems and methods can process radiograph data with one or more machine-learned models to generate a preliminary diagnosis. The preliminary diagnosis can be completed as a preliminary review or may provide diagnosis for patients who have a lack of accessibility to medical specialists. In some implementations, the machine-learned model can be specifically trained to process and classify data associated with mammography.

Another technical benefit of the systems and methods of the present disclosure is the ability to leverage machine-learned models to flag abnormalities in a patient's tissue. For example, a patient can receive a radiograph for a mammogram. The radiograph can be processed by one or more machine-learned models to generate a preliminary report on whether the patient has any abnormalities. Additionally, the systems and methods can provide the output in a universally displayable format that can allow medical professionals across a network to view the output in different environments that may not necessarily have mammogram specific displays (e.g., displays with a 1024 level grayscale range). In some implementations, the systems and methods can utilize cloud computing for efficient use of computing resources.

With reference now to the Figures, example embodiments of the present disclosure will be discussed in further detail.

### Example Devices and Systems

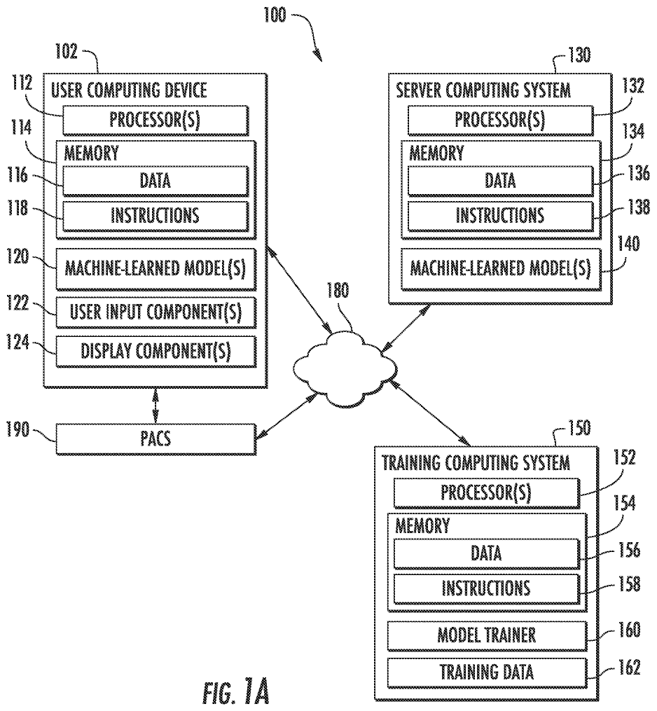

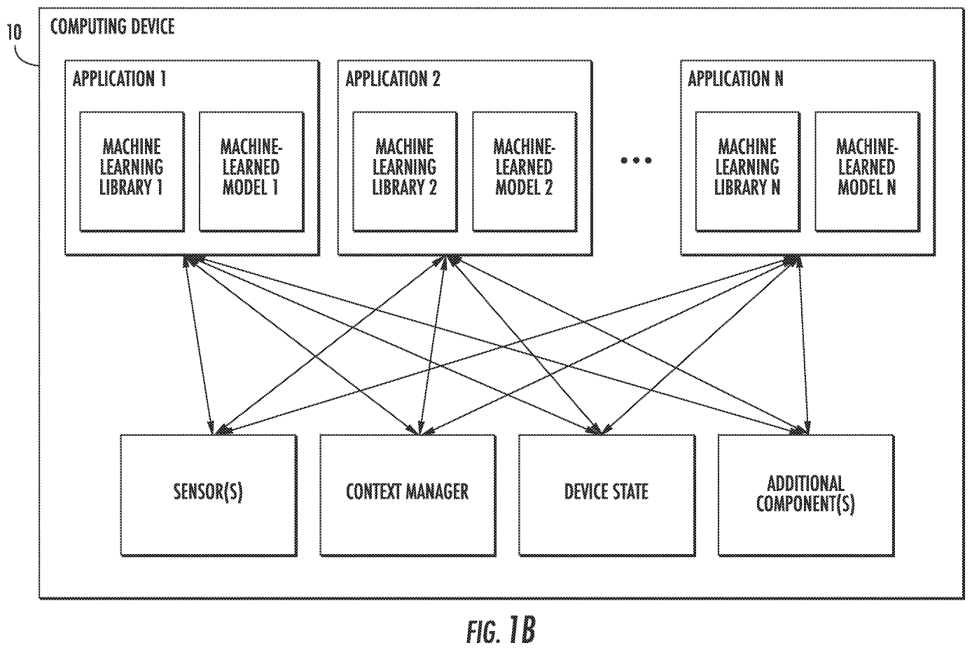

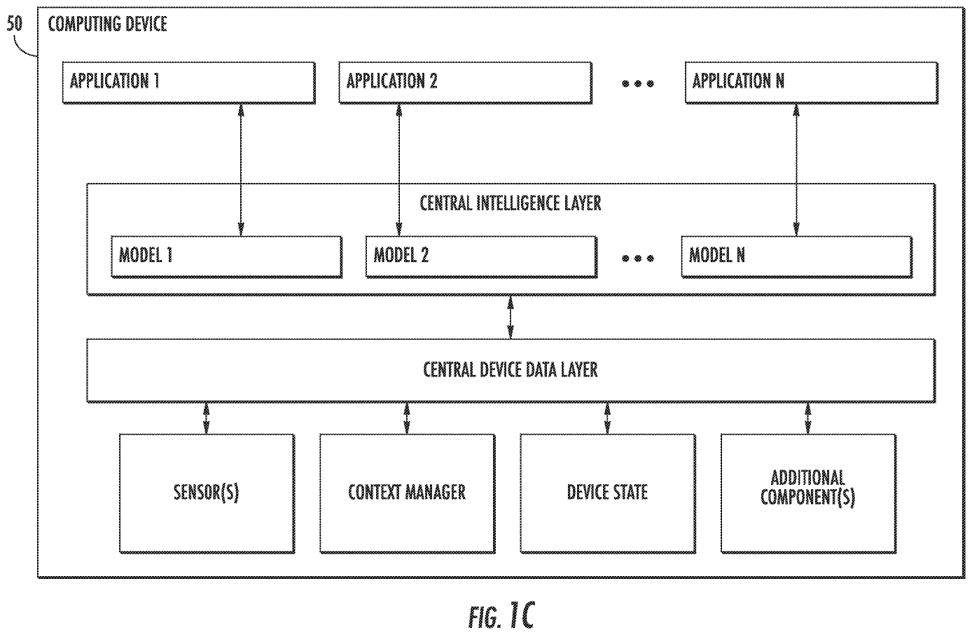

FIG. 1A depicts a block diagram of an example computing system 100 that performs visual representation output generation according to example embodiments of the present disclosure. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

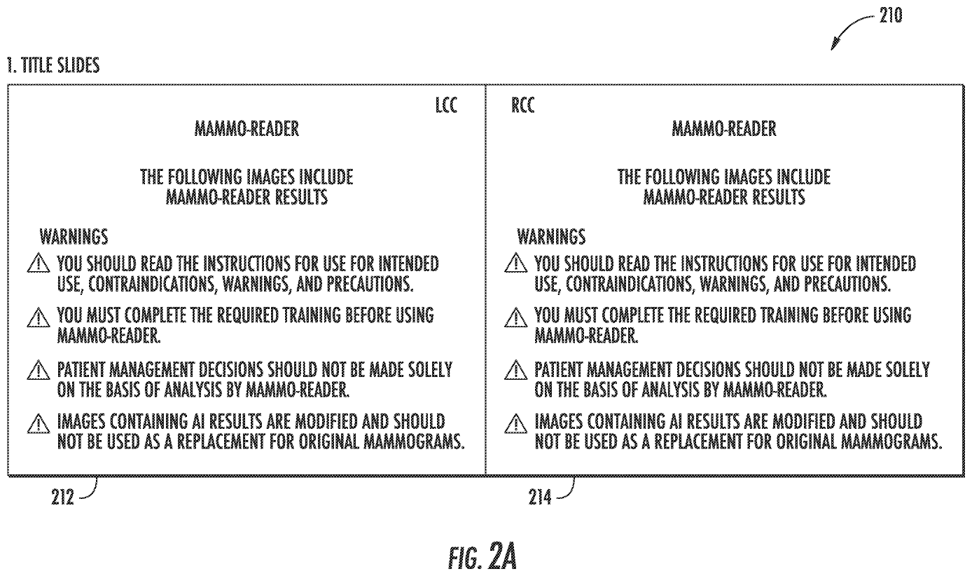

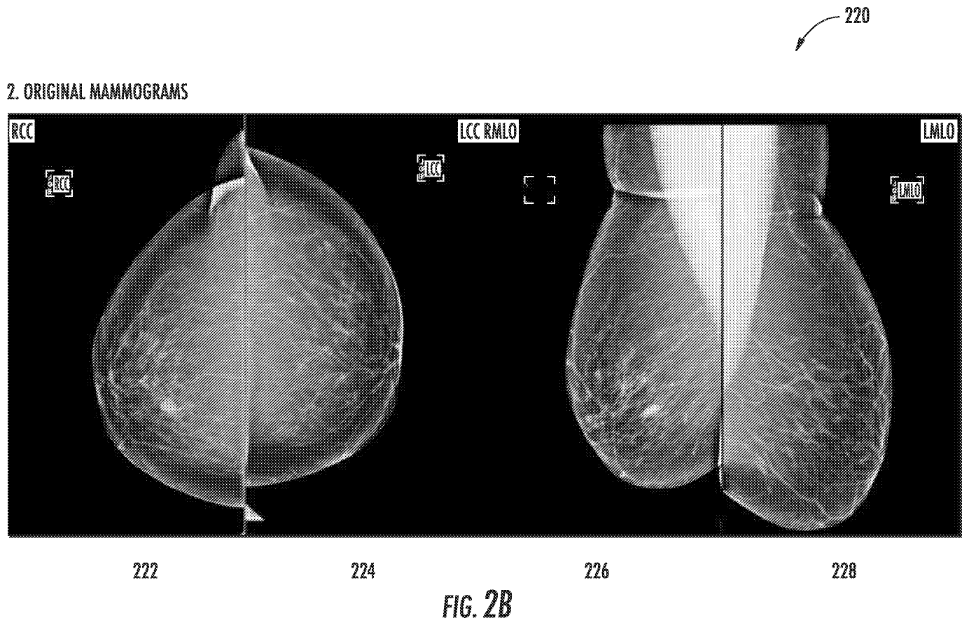

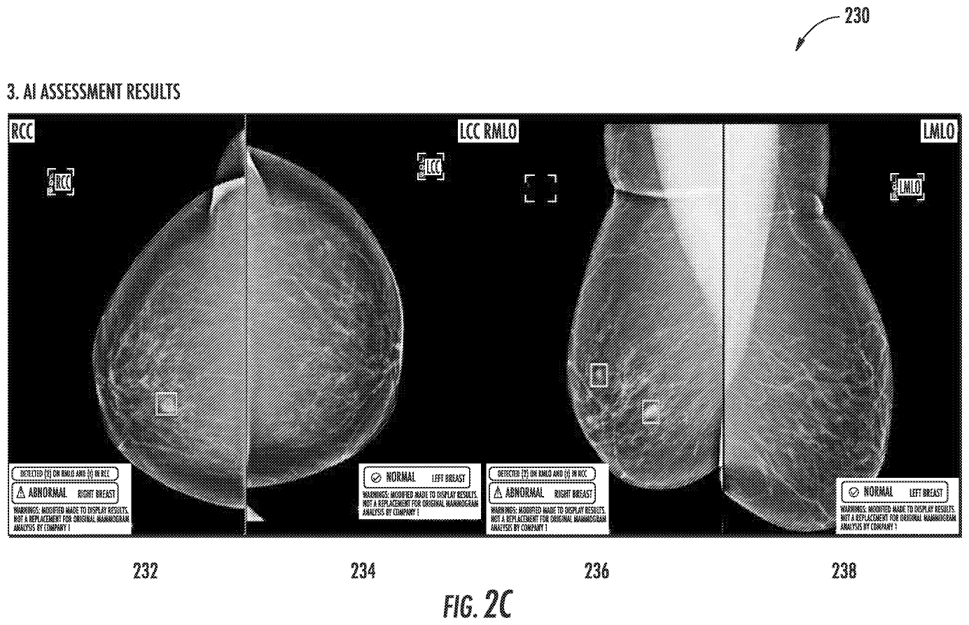

In some implementations, the user computing device 102 can store or include one or more machine-learned models 120. For example, the machine-learned models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Example machine-learned models 120 are discussed with reference to FIGS. 2A-5.

In some implementations, the one or more machine-learned models 120 can be received from the server computing system 130 over network 180, stored in the user computing device memory 114, and then used or otherwise implemented by the one or more processors 112. In some implementations, the user computing device 102 can implement multiple parallel instances of a single machine-learned model 120 (e.g., to perform parallel visual representation output generation across multiple instances of radiograph images).

More particularly, the one or more machine-learned models 120 can include a visual representation generation model, which may include one or more detection models, one or more segmentation models, one or more classification models, one or more augmentation models, and/or one or more other machine-learned models. The one or more machine-learned models 120 can include one or more EfficientNet models. The one or more machine-learned models can be trained and/or configured to detect, segment, and classify abnormal features in radiograph data (e.g., mammography image data).

Additionally or alternatively, one or more machine-learned models 140 can be included in or otherwise stored and implemented by the server computing system 130 that communicates with the user computing device 102 according to a client-server relationship. For example, the machine-learned models 140 can be implemented by the server computing system 140 as a portion of a web service (e.g., a preliminary breast cancer diagnosis service). Thus, one or more models 120 can be stored and implemented at the user computing device 102 and/or one or more models 140 can be stored and implemented at the server computing system 130.

The user computing device 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

Additionally and/or alternatively, the user computing device 102 can include one or more display components 124 to provide data for display. The user computing device 102 can include light emission display elements and/or can include technical capabilities for providing varying datasets for display.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned models 140. For example, the models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Example models 140 are discussed with reference to FIGS. 2A-5. In some implementations, the user computing device 102 may not include a machine-learned model 120 and may instead utilize the one or more machine-learned models 140 of the server computing system 130.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The one or more processors 152 can include a CPU, a GPU, and/or a TPU. For example, the GPU and/or the TPU can be utilized for training and inference and can help greatly increase performance. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the machine-learned models 120 and/or 140 based on a set of training data 162. The training data 162 can include, for example, a plurality of training radiograph images, a plurality of ground truth bounding boxes, a plurality of ground truth segmentation masks, a plurality of ground truth classifications, a plurality of training example outputs, and/or a plurality of training pixel datasets.

In some implementations, if the user has provided consent, the training examples can be provided by the user computing device 102. Thus, in such implementations, the model 120 provided to the user computing device 102 can be trained by the training computing system 150 on user-specific data received from the user computing device 102. In some instances, this process can be referred to as personalizing the model.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM hard disk or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

In some implementations, the example computing system 100 can include a picture archiving and communications system 190 that may store and/or provide access to data provided by the user computing device 102, the server computing system 130, and/or the training computing system 150 via a network 180 and/or a direct connection. The picture archiving and communications system 190 can include a medical imaging technology utilized by medical service providers to store and transmit reports and/or images.

The machine-learned models described in this specification may be used in a variety of tasks, applications, and/or use cases.

In some implementations, the input to the machine-learned model(s) of the present disclosure can be image data. The machine-learned model(s) can process the image data to generate an output. As an example, the machine-learned model(s) can process the image data to generate an image recognition output (e.g., a recognition of the image data, a latent embedding of the image data, an encoded representation of the image data, a hash of the image data, etc.). As another example, the machine-learned model(s) can process the image data to generate an image segmentation output. As another example, the machine-learned model(s) can process the image data to generate an image classification output. As another example, the machine-learned model(s) can process the image data to generate an image data modification output (e.g., an alteration of the image data, etc.). As another example, the machine-learned model(s) can process the image data to generate an encoded image data output (e.g., an encoded and/or compressed representation of the image data, etc.). As another example, the machine-learned model(s) can process the image data to generate an upscaled image data output. As another example, the machine-learned model(s) can process the image data to generate a prediction output.

In some implementations, the input to the machine-learned model(s) of the present disclosure can be text or natural language data. The machine-learned model(s) can process the text or natural language data to generate an output. As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language, etc.). As another example, the machine-learned model(s) can process the text or natural language data to generate a prediction output.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some implementations, the input to the machine-learned model(s) of the present disclosure can be statistical data. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the machine-learned model(s) can be configured to perform a task that includes encoding input data for reliable and/or efficient transmission or storage (and/or corresponding decoding). For example, the input includes visual data (e.g., one or more images or videos), the output comprises compressed visual data, and the task is a visual data compression task. In another example, the task may comprise generating an embedding for input data (e.g., input audio or visual data).

In some cases, the input includes visual data and the task is a computer vision task. In some cases, the input includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

FIG. 1A illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. In some of such implementations, the user computing device 102 can implement the model trainer 160 to personalize the models 120 based on user-specific data.

FIG. 1B depicts a block diagram of an example computing device 10 that performs according to example embodiments of the present disclosure. The computing device 10 can be a user computing device or a server computing device.

The computing device 10 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 1B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 1C depicts a block diagram of an example computing device 50 that performs according to example embodiments of the present disclosure. The computing device 50 can be a user computing device or a server computing device.

The computing device 50 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model (e.g., a model) can be provided for each application and managed by the central intelligence layer. In other implementations, two or more applications can share a single machine-learned model. For example, in some implementations, the central intelligence layer can provide a single model (e.g., a single model) for all of the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

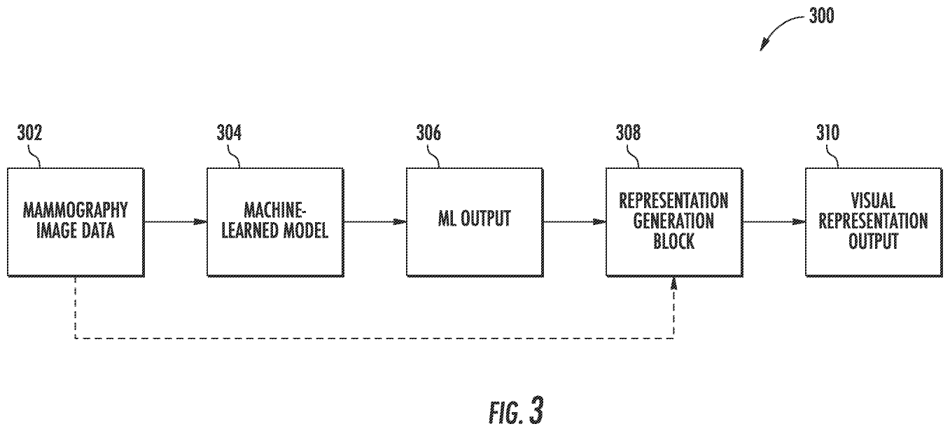

In some implementations, the computing system 100 can include utilizing one or more sensors to obtain and/or generate data. The data can then be obtained by and/or input into one or more applications that can then utilize the one or more machine-learned models to process the data and generate an output (e.g., as depicted in FIG. 3).

### Example UX and Model Arrangements

FIGS. 2A-2C depict illustrations of example user interface slides according to example embodiments of the present disclosure. For example, the user interface as disclosed herein can utilize a plurality of informational slides to provide a user with instructions (e.g., FIG. 2A), unannotated radiograph images (e.g., FIG. 2B), and/or annotated radiograph images (e.g., FIG. 2C).

In FIG. 2A, one or more title slides 210 (e.g., instructional slides) can be provided for display. The one or more title slides 210 may be provided for display in a dual format with a left slide 212 and a right slide 214. The left slide 212 and the right slide 214 can include the same instructions and/or may differ. The one or more title slides 210 can include a warning related to the model outputs, the information about to be provided, and/or about the accuracy of the outputs. In some implementations, the one or more title slides 210 may include information on how to interpret the later slides.

In FIG. 2B, one or more original mammogram slides 220 can be provided for display. The one or more original mammogram slides 220 can be descriptive of unannotated radiograph images. In some implementations, the one or more original mammogram slides 220 can be generated by generating display-adjusted radiograph images that can be provided for display on display devices with 256-level grayscale capabilities. Additionally and/or alternatively, hanging protocols can be utilized to determine an orientation and location of the radiograph images in the user interface. For example, from left to right, the user interface may display the right craniocaudal view 222, the left craniocaudal view 224, the right mediolateral oblique view 226, and/or the left mediolateral oblique view 228. The orientation may be uniform across instances of processing and display.

In FIG. 2C, one or more artificial intelligence assessment result slides 230 can be provided for display. The one or more artificial intelligence assessment result slides 230 can be descriptive of annotated radiograph images. In some implementations, the one or more artificial intelligence assessment result slides 230 can be generated by generating display-adjusted radiograph images that can be provided for display on display devices with 256-level grayscale capabilities. Additionally and/or alternatively, hanging protocols can be utilized to determine an orientation and location of the radiograph images in the user interface. For example, from left to right, the user interface may display the right craniocaudal view 232, the left craniocaudal view 234, the right mediolateral oblique view 236, and/or the left mediolateral oblique view 238. The orientation may be uniform across instances of processing and display. The radiograph images may be annotated with one or more user interface elements that indicate regions of interest with a high probability of abnormal tissue (e.g., tissue determined to be descriptive of potential cancer cells). The one or more user interface elements can include rectangles and may include a size and/or dimensions that may be dependent on the size and dimensions of the respective region of interest. Additionally and/or alternatively, each view may include additional information, which can include text that indicates an overall classification for the image (e.g., the image includes normal tissue, the image includes abnormal tissue, and/or the image includes obstructed data), an indicator of the respective body part associated with the image, a number of regions of interest, a confidence level of the preliminary diagnosis, a disclaimer, and/or other descriptive data.

FIG. 3 depicts a block diagram of an example visual representation generation model 300 according to example embodiments of the present disclosure. In some implementations, the visual representation generation model 300 is trained to receive a set of input data (e.g., mammography image data 302) descriptive of one or more radiograph images associated with an individual and, as a result of receipt of the input data, provide output data (e.g., a visual representation output 310) that can include one or more annotated radiograph images. Thus, in some implementations, the visual representation generation model 300 can include a machine-learned model 304 that is operable to detect, segment, and classify regions of interest in radiograph data.

For example, the visual representation generation model 300 can receive mammography image data 302 as input. The mammography image data 302 can be processed with a machine learned model 304 (e.g., one or more detection models, one or more segmentation models, one or more classification models, and/or one or more augmentation models) to generate a machine-learned model output 306 (e.g., one or more bounding boxes, one or more segmentation masks, one or more image patches, one or more classifications, one or more embeddings, and/or one or more scalable vector graphic datasets). The machine-learned model output 306 and/or the mammography image data 302 can then be processed by a representation generation block 308 to generate the visual representation output 310. The representation generation block 308 may adjust the color values of the radiograph images to allow for display on a broad variety of devices. Additionally and/or alternatively, the representation generation block 308 may utilize the machine-learned model output 306 to render user interface elements superimposed over the radiograph images to indicate regions of interest. One or more other user interface elements may be rendered in one or more other regions of the radiograph images.

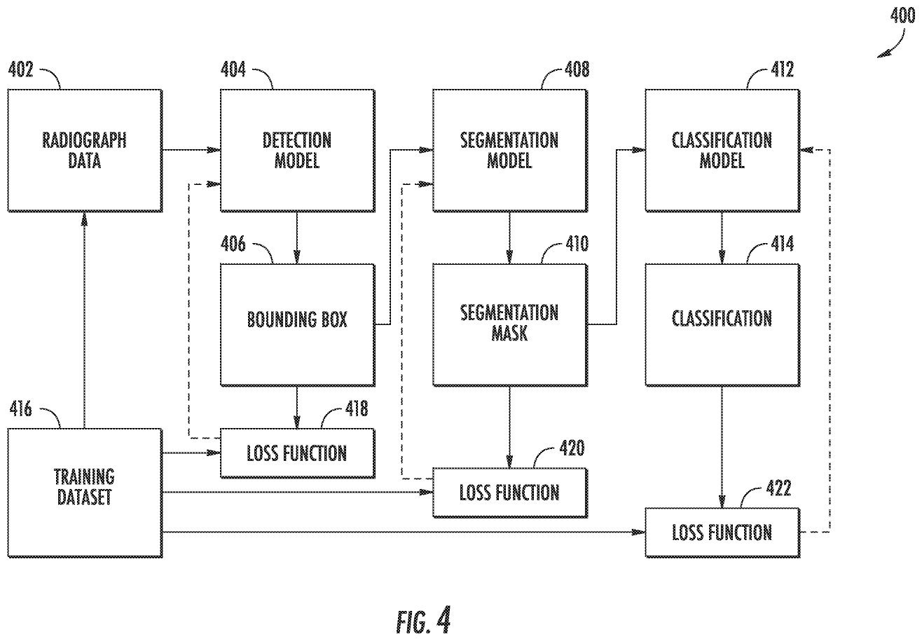

FIG. 4 depicts a block diagram of an example machine-learned model training 400 according to example embodiments of the present disclosure. The machine-learned model training 400 can include learning a plurality of parameters for a plurality of machine-learned models, which can include a detection model 404, a segmentation model 408, and/or a classification model 412. For example, a training dataset 416 can be obtained. The training dataset 416 can include example radiograph data 402, training bounding boxes (e.g., ground truth bounding boxes), training segmentation masks (e.g., ground truth segmentation masks), training classification labels (e.g., ground truth classification labels), and/or training visual representation data.

The example radiograph data 402 may be obtained and processed with a detection model 404 to generate one or more bounding boxes 406 that may indicate one or more detected abnormal features that may be regions of interest. The radiograph data 402 and/or the one or more bounding boxes 406 may be processed by a segmentation model 408 to generate one or more segmentation masks 410, which may be utilized to segment the regions of interest from the rest of the radiograph data 402. The segmented data can then be processed with a classification model 412 to generate one or more classifications 414 (e.g., one or more classifications descriptive of whether the particular region of interest includes normal tissue, cancer tissue, and/or other abnormal tissue).

One or more loss functions (e.g., 418, 420, and/or 422) can be utilized to evaluate the outputs to generate a gradient descent that can be utilized to adjust the parameters of one or more of the machine-learned models. For example, the one or more bounding boxes 406 and one or more training bounding boxes of the training dataset 416 can be utilized to evaluate a loss function 418 to generate a detection gradient descent that can be backpropagated to the detection model 404 to adjust one or more parameters of the detection model 404. Additionally and/or alternatively, the one or more segmentations masks 410 and one or more training segmentation masks of the training dataset 416 can be utilized to evaluate a loss function 420 to generate a segmentation gradient descent that can be backpropagated to the segmentation model 408 to adjust one or more parameters of the segmentation model 408. In some implementations, the one or more classifications 410 and one or more training classifications of the training dataset 416 can be utilized to evaluate a loss function 422 to generate a classification gradient descent that can be backpropagated to the classification model 412 to adjust one or more parameters of the classification model 412.

In some implementations, a single loss function may be utilized to evaluate the outputs of the plurality of models. For example, the visual representation output (and/or another output) can be evaluated against a ground truth output to evaluate the detection, segmentation, and/or classification. In some implementations, the loss function may include one or more terms for each respective model.

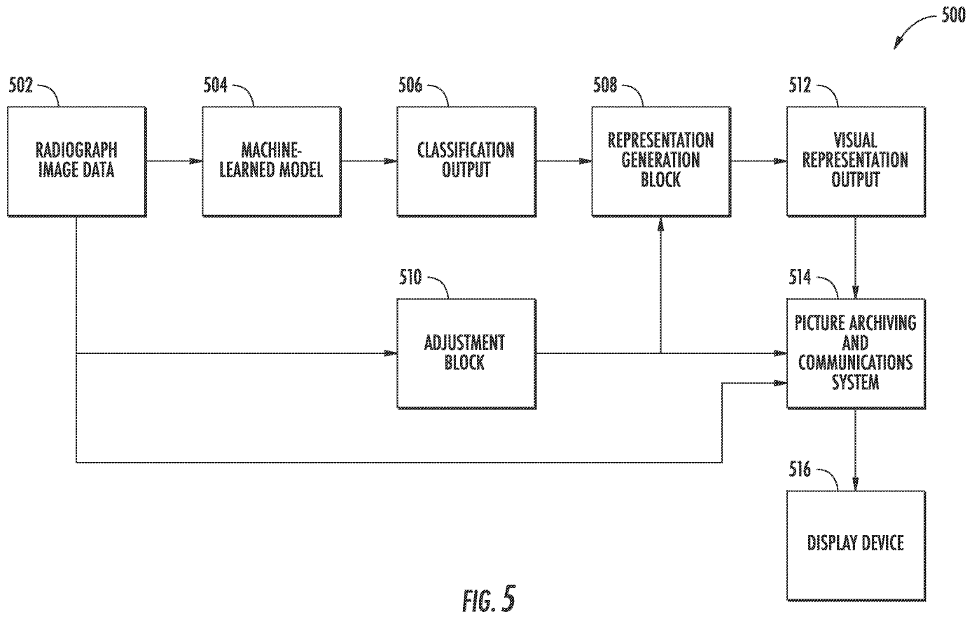

FIG. 5 depicts a block diagram of an example output generation and display system 500 according to example embodiments of the present disclosure. In particular, radiograph image data 502 can be obtained. The radiograph image data 502 can be descriptive of one or more radiographs (e.g., x-rays) associated with a particular individual. The radiographs can depict internal data associated with one or more body parts. The radiograph image data 502 can be processed with a machine-learned model 504 (e.g., an EfficientNet model, a convolutional neural network, a recurrent neural network, a self-attention model, and/or a transformer model) to generate a classification output 506. The classification output 506 can be descriptive of one or more preliminary diagnoses (e.g., a cancer diagnosis and/or a general abnormality diagnosis) for each respective region of interest and/or for the dataset as a whole.

The classification output 506 can then be processed with a representation generation block 508 to generate a visual representation output 512. In some implementations, the radiograph image data 502 can be processed with an adjustment block to generate a broadly displayable version of the radiograph images, which can then be processed with the classification output 506 by the representation generation block 508 to generate the visual representation output 512.

The visual representation output 512, the radiograph image data 502, and/or broadly displayable version of the radiograph images can then be transmitted to and/or stored with a picture archiving and communications system 514. One or more display devices 516 can then communicate with the picture archiving and communications system 514 to obtain and provide for display one or more of the visual representation output 512 and/or broadly displayable version of the radiograph images. The one or more display devices 516 can include a display device that may not be optimized for radiograph images.

### Example Methods

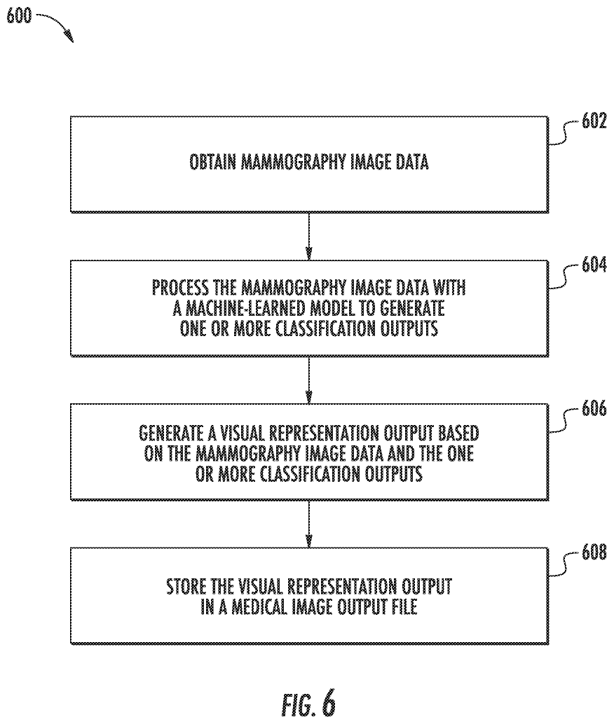

FIG. 6 depicts a flow chart diagram of an example method to perform according to example embodiments of the present disclosure. Although FIG. 6 depicts steps performed in a particular order for purposes of illustration and discussion, the methods of the present disclosure are not limited to the particularly illustrated order or arrangement. The various steps of the method 600 can be omitted, rearranged, combined, and/or adapted in various ways without deviating from the scope of the present disclosure.

At 602, a computing system can obtain mammography image data. The mammography image data can include radiograph data associated with an individual. In some implementations, the mammography image data can be descriptive of one or more radiograph images. The mammography image data can be descriptive of a left mediolateral oblique view, a right mediolateral oblique view, a left craniocaudal view, and a right craniocaudal view.

At 604, the computing system can process the mammography image data with a machine-learned model to generate one or more classification outputs. The machine-learned model may have been trained to detect and classify an abnormality in the radiograph data. The abnormality can be associated with a predicted cancer classification. In some implementations, the one or more classification outputs can be descriptive of one or more detected abnormalities. Additionally and/or alternatively, processing the mammography image data with the machine-learned model to generate the one or more classification outputs can include cloud computing. In some implementations, the machine-learned model can process the mammography image data to identify regions of interest, segment the regions of interest, and classify the features in the regions of interest. The regions of interest may be identified by a detection model trained to determine one or more features for classification. The region encompassing the feature can be segmented then classified. The machine-learned model can include one or more detection models, one or more segmentation models, one or more classification models, one or more augmentation models, and/or one or more semantic understanding models. The machine-learned model may be trained on a training dataset comprising a plurality of training radiograph images associated with breast tissue of an individual. The plurality of training radiograph images may include ground truth healthy images and/or ground truth cancer images (e.g., images with one or more abnormal features associated with cancer cells). The training dataset may include a plurality of ground truth labels and/or a plurality of example segmentation masks.

At 606, the computing system can generate a visual representation output based on the mammography image data and the one or more classification outputs. The visual representation output can include one or more annotated radiograph images. The one or more annotated radiograph images can be descriptive of the one or more radiograph images with one or more annotations associated with the one or more detected abnormalities. Additionally and/or alternatively, the visual representation output can include text data associated with the one or more classification outputs. The one or more annotations can be specifically generated for each respective detected abnormality. For example the size and/or shape of each annotation may be specific to each respective detected abnormality. In some implementations, the size and/or shape may be proportional to the size and/or shape of the detected abnormal cells. The one or more annotations can include a first rectangle superimposed over a second rectangle with thicker borders. In some implementations, the visual representation output can include one or more grayscale images.

In some implementations, the one or more annotations can indicate one or more portions of the one or more radiograph images determined to include a region of interest. The text data can include text information descriptive of an abnormality classification. The abnormality classification can be descriptive of the one or more radiograph images including pixel data associated with a tissue abnormality. Additionally and/or alternatively, the text data can include text information descriptive of a number of detected abnormalities. The text data can include a text label indicating the breast where the image came from (left or right) and/or the artificial intelligence assessment result for that breast (e.g., a binary score computed from this image and/or another image from the same breast: normal or abnormal). A breast may be considered abnormal if the machine-learned model reports at least one region of interest, with the risk score above the threshold on any image of that breast. Otherwise, the breast may receive a normal score. The text data may include a text warning indicating that the visual representation output is not an original image and should not be used alone for making clinical decisions. Additionally and/or alternatively, the text data can include a text label indicating the number of regions of interest on a given image and/or other images of the same breast. In some implementations, the one or more annotations can include one or more boxes. The one or more boxes can indicate one or more regions of interest associated with one or more detected abnormalities. In some implementations, a size, a width, and a length of the one or more annotations may be determined based on determined dimensions of the one or more detected abnormalities.

At 608, the computing system can store the visual representation output in a medical image output file. The computing system may provide the visual representation output to a display computing system for display. The display computing system may include a general purpose display (e.g., a standard light-emitting diode display, an organic light-emitting diode display, a touch screen monitor, a plasma screen monitor, and/or another monitor type). The visual representation output may be of a different color range than the one or more radiograph images.

In some implementations, the one or more radiograph images can include a first number of gray scales. The one or more annotated radiograph images can include a second number of gray scales. Additionally and/or alternatively, the second number can be less than the first number. The second number may be optimized for universal display on a plurality of different display types.

In some implementations, the computing system can transmit the medical image output file, including the visual representation output, to a picture archiving and communication system for storage. The computing system may provide the visual representation output for display via a display computing device. The medical image output file can be configured to instruct the display computing device to display the visual representation output within a set grayscale range. The picture archiving and communication system can archive the mammography image data and/or the visual representation output. The archived data can then be obtained and displayed in response to one or more requests. The archived data may be accessed via a plurality of computing devices across a network.

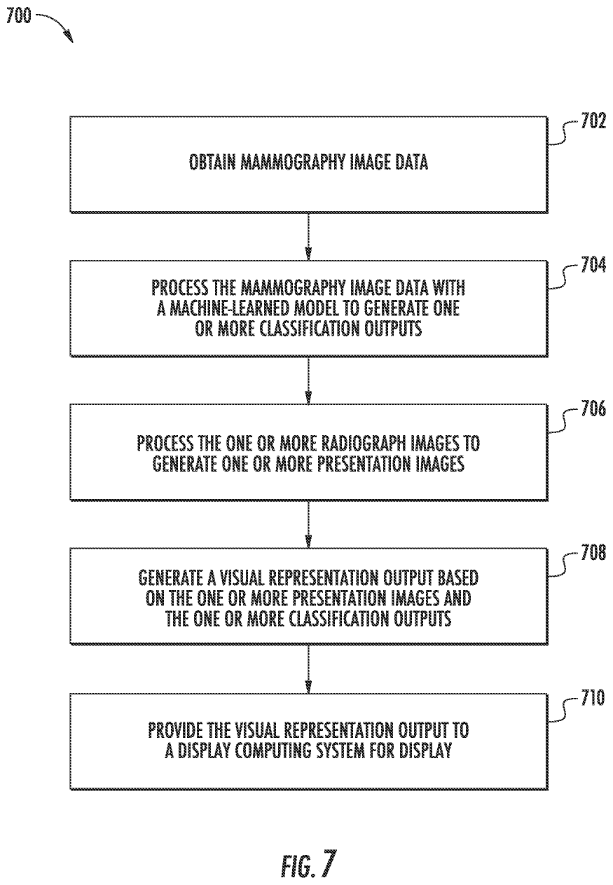

FIG. 7 depicts a flow chart diagram of an example method to perform according to example embodiments of the present disclosure. Although FIG. 7 depicts steps performed in a particular order for purposes of illustration and discussion, the methods of the present disclosure are not limited to the particularly illustrated order or arrangement. The various steps of the method 700 can be omitted, rearranged, combined, and/or adapted in various ways without deviating from the scope of the present disclosure.

At 702, a computing system can obtain mammography image data. The mammography image data can include radiograph data associated with an individual. In some implementations, the mammography image data can be descriptive of one or more radiograph images. The one or more radiograph images can be descriptive of an x-ray of one or more angles of an individual's breast. The mammography image data may include metadata associated with the user, which may include an individual's age, weight, and/or other biological information.

At 704, the computing system can process the mammography image data with a machine-learned model to generate one or more classification outputs. The machine-learned model may have been trained to detect and classify an abnormality in the radiograph data. The abnormality can be associated with a predicted cancer classification. The one or more classification outputs can be descriptive of one or more detected abnormalities. The one or more classification outputs can be associated with a preliminary diagnosis associated with processed radiograph data (e.g., a preliminary cancer diagnosis). In some implementations, the machine-learned model can be trained to process and provide a preliminary diagnosis associated with one or more other body parts and/or one or more other diagnostic options (e.g., a preliminary benign cyst diagnosis, a preliminary anterior cruciate ligament (ACL) tear diagnosis for a knee, and/or a stroke by processing brain imagery). The systems and methods may generate an optimized output based on the machine-learned model output that can effectively communicate a preliminary diagnosis to a radiologist, another medical professional, an insurance provider, and/or a patient.

In some implementations, the machine-learned model can include a feature detection model trained to identify regions of interest associated with abnormal features in a radiograph. The feature detection model can generate one or more bounding boxes. In some implementations, the machine-learned model can include a segmentation model trained to process a radiograph and segment a region of interest from the radiograph. Additionally and/or alternatively, the machine-learned model can include a classification model trained to process a region of interest to generate an abnormality classification.

At 706, the computing system can process the one or more radiograph images to generate one or more presentation images. The one or more presentation images can be descriptive of the one or more radiograph images augmented for presentation on a visual display. The one or more presentation images may encode bounding boxes to use a lightest available pixel value (e.g. the maximum pixel value).

At 708, the computing system can generate a visual representation output based on the one or more presentation images and the one or more classification outputs. The visual representation output can include one or more annotated radiograph images. In some implementations, the one or more annotated radiograph images can be descriptive of the one or more presentation images with one or more annotations associated with the one or more detected abnormalities.

At 710, the computing system can provide the visual representation output to a display computing system for display. In some implementations, the computing system can store the mammography image data and the visual representation output in association with one or more individual identifiers that identify the individual. The mammography image data and the visual representation output may be stored in a medical image output file. The medical image output file can be formatted to provide the one or more radiograph images and the visual representation output in a displayable format for a plurality of display types.

Additionally and/or alternatively, the computing system can present a title slide (e.g., via a DICOM image output, which can include DICOM tags for enabling the hanging protocols to arrange the images (e.g., the title slide, the raw image, and the annotated image) in a particular order). The title slide can include one or more instructions. In response to obtaining the mammography image data, the systems and methods may present the one or more radiograph images via the user interface. In some implementations, the visual representation output can be provided for display via the user interface. Through the use of hanging protocols in the viewer, the title slide may be uniformly presented prior to any AI output slides and raw images may be uniformly presented prior to any AI annotated versions of those raw images.

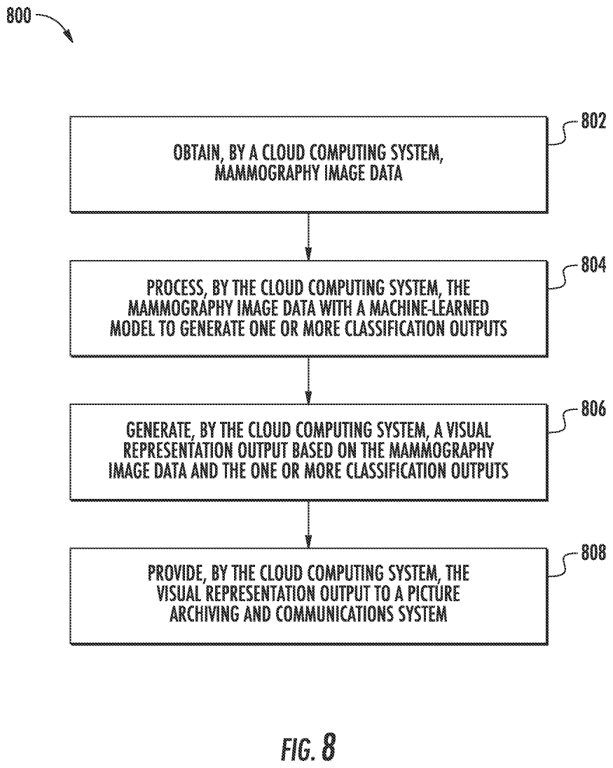

FIG. 8 depicts a flow chart diagram of an example method to perform according to example embodiments of the present disclosure. Although FIG. 8 depicts steps performed in a particular order for purposes of illustration and discussion, the methods of the present disclosure are not limited to the particularly illustrated order or arrangement. The various steps of the method 800 can be omitted, rearranged, combined, and/or adapted in various ways without deviating from the scope of the present disclosure.

At 802, a computing system can obtain, by a cloud computing system, mammography image data. The mammography image data can include x-ray data associated with breast tissue of an individual. In some implementations, the mammography image data can be descriptive of one or more radiograph images. The cloud computing system can include one or more server computing systems. The one or more server computing systems can include the one or more processors. The one or more radiograph images may be digital imaging and communications in medicine standard (DICOM) images.

At 804, the computing system can process, by the cloud computing system, the mammography image data with a machine-learned model to generate one or more classification outputs. The machine-learned model may be trained to detect and classify an abnormality in the x-ray data. The abnormality can be associated with a predicted cancer classification. In some implementations, the one or more classification outputs can be descriptive of one or more detected abnormalities. The mammography image data may be processed in black-and-white (e.g., in grayscale). The mammography image data can include one or more high fidelity images that may be obtained and/or stored in a central computing system.

At 806, the computing system can generate, by the cloud computing system, a visual representation output based on the mammography image data and the one or more classification outputs. The visual representation output can include one or more annotated radiograph images. The one or more annotated radiograph images can be descriptive of the one or more radiograph images with one or more annotations associated with the one or more detected abnormalities. The visual representation may include only positive abnormality classification and may remove and/or determine not to include any annotations associated with negative classifications for one or more determined regions of interest. In some implementations, the visual representation output can include text descriptive of a number of detected abnormalities and/or one or more region of interest classifications and/or one or more image classifications. The systems and methods may generate a visual representation output for each obtained radiograph image of a plurality of radiograph images. In some implementations, the annotations may be overlapping, which may indicate overlapping regions of interest. Alternatively and/or additionally, the annotation associated with a higher confidence score of abnormality may be displayed with the other annotation removed. Alternatively and/or additionally, the larger annotation associated with a larger bounding box may be kept. The visual representation output may be generated based on a particular resolution, bit type or amount, and/or color range associated with one or more hardware devices.

At 808, the computing system can provide, by the cloud computing system, the visual representation output to a picture archiving and communications system. The visual representation output can be optimized for the picture archiving and communications system. The visual representation output can be generated to be universally compatible for a plurality of different types of display. The systems and methods may run end to end. The systems and methods may be utilized to provide a preliminary diagnosis without the cost of a dedicated high processing power workstation for each individual testing unit. The systems and methods may utilize cloud computing to allow the use of the processing power of one or more server computing systems, which may lead to less costs and less dedicated processing components and/or devices. The metadata for the mammography image data may be utilized for uniform posing of the outputs (e.g., corrected orientation for uniformity based one hanging protocols). The picture archiving and communications system can include a plurality of computing devices with different displays, different interfaces, and/or technical capabilities.

In some implementations, the cloud computing system can transmit, from the picture archiving and communications system to a display device, the visual representation output.

Additionally and/or alternatively, the one or more radiograph images can include a plurality of pixels in a first range of 1024 shades of gray, and the one or more annotated radiograph images can include pixel data in a second range. The second range can include less shades than the first range.

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

1. A computing system for providing a mammography device output, the system comprising:
one or more processors; and
one or more non-transitory computer-readable media that collectively store instructions that, when executed by the one or more processors, cause the computing system to perform operations, the operations comprising:
obtaining mammography image data, wherein the mammography image data comprises radiograph data associated with an individual, and wherein the mammography image data is descriptive of one or more radiograph images;
processing the mammography image data with a machine-learned model to generate one or more classification outputs, wherein the machine-learned model was trained to detect and classify an abnormality in the radiograph data, wherein the abnormality is associated with a predicted cancer classification, and wherein the one or more classification outputs are descriptive of one or more detected abnormalities;
generating a visual representation output based on the mammography image data and the one or more classification outputs, wherein the visual representation output comprises one or more annotated radiograph images, wherein the one or more annotated radiograph images are descriptive of the one or more radiograph images with one or more annotations associated with the one or more detected abnormalities, and wherein the visual representation output comprises text data associated with the one or more classification outputs; and
storing the visual representation output in a medical image output file.

2. The system of claim 1, wherein the one or more annotations indicate one or more portions of the one or more radiograph images determined to include a region of interest.

3. The system of claim 1, wherein the text data comprises text information descriptive of an abnormality classification, wherein the abnormality classification is descriptive of the one or more radiograph images comprising pixel data associated with a tissue abnormality.

4. The system of claim 1, wherein the text data comprises text information descriptive of a number of detected abnormalities.

5. The system of claim 1, wherein the one or more radiograph images comprise a first bit depth, wherein the one or more annotated radiograph images comprise a second bit depth, and wherein the second bit depth is smaller than the first bit depth.

6. The system of claim 1, wherein processing the mammography image data with the machine-learned model to generate the one or more classification outputs comprises cloud computing.

7. The system of claim 1, wherein the operations further comprise:
transmitting the medical image output file to a picture archiving and communication system; and
providing the visual representation output for display via a display computing device, wherein the medical image output file is configured to instruct the display computing device to display the visual representation output within a set grayscale range.

8. The system of claim 1, wherein the one or more annotations comprise one or more boxes, wherein the one or more boxes indicate one or more regions of interests associated with one or more detected abnormalities.

9. The system of claim 1, wherein a size, a width, and a length of the one or more annotations are determined based on determined dimensions of the one or more detected abnormalities.

10. The system of claim 1, wherein the mammography image data is descriptive of a left mediolateral oblique view, a right mediolateral oblique view, a left craniocaudal view, and a right craniocaudal view.

11. One or more non-transitory computer-readable media that collectively store instructions that, when executed by one or more computing devices, cause the one or more computing devices to perform operations, the operations comprising:
obtaining mammography image data, wherein the mammography image data comprises radiograph data associated with an individual, and wherein the mammography image data is descriptive of one or more radiograph images;
processing the mammography image data with a machine-learned model to generate one or more classification outputs, wherein the machine-learned model was trained to detect and classify an abnormality in the radiograph data, wherein the abnormality is associated with a predicted cancer classification, and wherein the one or more classification outputs are descriptive of one or more detected abnormalities;
processing the one or more radiograph images to generate one or more presentation images, wherein the one or more presentation images are descriptive of the one or more radiograph images augmented for presentation on a visual display;
generating a visual representation output based on the one or more presentation images and the one or more classification outputs, wherein the visual representation output comprises one or more annotated radiograph images, wherein the one or more annotated radiograph images are descriptive of the one or more presentation images with one or more annotations associated with the one or more detected abnormalities; and
providing the visual representation output to a display computing system for display.

12. The one or more non-transitory computer-readable media of claim 11, wherein the machine-learned model comprises a feature detection model trained to identify regions of interest associated with abnormal features in a radiograph, and wherein the feature detection model generates one or more bounding boxes.

13. The one or more non-transitory computer-readable media of claim 11, wherein the machine-learned model comprises a segmentation model trained to process a radiograph and segment a region of interest from the radiograph.

14. The one or more non-transitory computer-readable media of claim 11, wherein the machine-learned model comprises a classification model trained to process a region of interest to generate an abnormality classification.

15. The one or more non-transitory computer-readable media of claim 11, the operations further comprising:
storing the mammography image data and the visual representation output in association with one or more individual identifiers that identify the individual, wherein the mammography image data and the visual representation output are stored in a medical image output file, wherein the medical image output file is formatted to provide the one or more radiograph images and the visual representation output in a displayable format for a plurality of display types.

16. The one or more non-transitory computer-readable media of claim 11, the operations further comprising:
presenting a title slide, wherein title slide comprises one or more instructions;
in response to obtaining the mammography image data, presenting the one or more radiograph images via the user interface; and
wherein the visual representation output is provided for display via the user interface.

17. A computer-implemented method for providing a mammography device output, the method comprising:
obtaining, by a cloud computing system comprising one or more processors, mammography image data, wherein the mammography image data comprises x-ray data associated with breast tissue of an individual, and wherein the mammography image data is descriptive of one or more radiograph images;
processing, by the cloud computing system, the mammography image data with a machine-learned model to generate one or more classification outputs, wherein the machine-learned model was trained to detect and classify an abnormality in the x-ray data, wherein the abnormality is associated with a predicted cancer classification, and wherein the one or more classification outputs are descriptive of one or more detected abnormalities;
generating, by the cloud computing system, a visual representation output based on the mammography image data and the one or more classification outputs, wherein the visual representation output comprises one or more annotated radiograph images, wherein the one or more annotated radiograph images are descriptive of the one or more radiograph images with one or more annotations associated with the one or more detected abnormalities; and
providing, by the cloud computing system, the visual representation output to a picture archiving and communications system.

18. The method of claim 17, wherein the cloud computing system comprises one or more server computing systems, wherein the one or more server computing systems comprise the one or more processors.

19. The method of claim 17, further comprising:
transmitting, from the picture archiving and communications system to a display device, the visual representation output.

20. The method of claim 17, wherein the one or more radiograph images comprise a plurality of pixels in a first range of at least one of 1024 shades of gray or 4096 shades of gray, and wherein the one or more annotated radiograph images comprise pixel data in a second range, wherein the second range comprises less shades than the first range.
